(19) United States
(12) Patent Application Publication
(10) Pub. No.: US 2019/0290478 A1
(43) Pub. Date: Sep. 26, 2019

(54) MEDICINAL HERBAL COMPOSITION FOR EYE APPLICATION

(71) Applicant: I-Szu Hsia, San Jose, CA (US)

(72) Inventor: I-Szu Hsia, San Jose, CA (US)

(21) Appl. No.: 16/362,654

(22) Filed: Mar. 24, 2019

Related U.S. Application Data
(60) Provisional application No. 62/647,655, filed on Mar. 24, 2018.

(51) Int. Cl. A61F 7/00; A61F 7/02; A61K 36/815; A61K 36/287; A61P 27/02; A61K 9/00

(52) U.S. Cl. CPC A61F 7/0085; A61F 7/007; A61F 7/02; H04W 88/02; A61K 36/287; A61P 27/02; A61K 9/0048; A61K 36/815

(57) ABSTRACT

An apparatus that includes a frame is for securely holding the medication pad with a moisturized medication compartment held by a holder. The frame holds the medication pad to apply the medication to the eyes wherein the frame further includes a controllable heating element for heating the medication pad. The medication pad further comprises a moisturized compartment containing moisturized extracts of herbal medication for generating and applying a medication steam to the eyes when the heating element is controlled to apply heat to the medication pad.

### CROSS REFERENCE TO RELATED APPLICATIONS

This Application is a Non-provisional Application and claims the Priority Date of a previously filed Provisional Application 62/645,675 filed on Mar. 24, 2018 by the Applicant of this application. The disclosures made in Application 62/645,675 are hereby incorporated herein by reference.

### FIELD OF THE INVENTION

This invention relates to herbal compositions. More specifically, this invention relates to delivers extracts of goji berry and chrysanthemum via a steam-generating heating device. The herbal steam helps to lubricate and nourish and promotes blood circulation in the eye to alleviate dry eye symptoms.

### BACKGROUND OF THE INVENTION

There are many causes that could lead to reduction in vision (blurred vision, hazy vision, indistinct vision, dry eye) and eyestrain are known as near (reflex) triad. It is common known that the near triad includes (1) reduction of accommodation, (2) reduction of pupillary response, and (3) convergence response abnormality. The reduction of accommodation is attributed to hypertonicity of the ciliary muscle or lens hardening and the reduction in pupillary response is caused by a decrease in pupillary constriction rate or decrease in response speed. Additionally, the convergence response abnormality is attributed to an abnormal change of pupil position of both eyes. One of the known causes of dry eye is insufficient tear film formation on the corneal surface due to obstruction of meibomian glands.

Specifically, the dry eye disease (DED) is more formally known as keratoconjunctivitis sicca (KCS), is a common ophthalmologic disorder causing ocular discomfort in daily life. It occurs when the quantity and/or quality of tears fails to keep the surface of the eye adequately lubricated. Dry eye causes a scratchy sensation or the feeling that something is in the eye. Other symptoms include stinging or burning, episodes of excess tearing that follow periods of dryness, discharge, pain, and redness in the eye. People with dry eye may also feel as if their eyelids are heavy and may experience blurred vision.

Causes of reduction in vision (blurred vision, hazy vision, indistinct vision, dry eye) and eyestrain are known as near (reflex) triad. The near triad includes (1) reduction of accommodation, (2) reduction of pupillary response, and (3) convergence response abnormality. Reduction of accommodation is attributed to hypertonicity of the ciliary muscle or lens hardening. Reduction in pupillary response is caused by a decrease in pupillary constriction rate or decrease in response speed. Convergence response abnormality is attributed to an abnormal change of pupil position of both eyes. One of the known causes of dry eye is insufficient tear film formation on the corneal surface due to obstruction of meibomian glands.

Several patented documents and published patent applications disclosed vision improving device including U.S. Pat. Nos. 6,823,860 and 8,333,793 that supplies steam to the eyes and the surrounding areas in order to improve the function of accommodation muscles. The steam that introduced to the ciliary muscle and the eyelids generally improve the visual acuity of pseudomyopic eyes or aged eyes. The disclosed vision improving devices are designed to release steam from its surface at a controlled elevated temperature. The purposes are to recover the vision or reduce bleariness or blur in vision associated with dysfunction of accommodation muscles or accommodative spasm.

However, the heat and steam alone are not sufficiently effective to relief and cure the dry eye problems. There is still a need to provide more effective and convenient methods and devices to solve the dry eye problems.

### SUMMARY OF THE INVENTION

An object of this invention is to provide a more convenient and effective way to deal with the dry eyes by adding a medicinal herbal composition such as chrysanthemum and Goji berry through the steam-generating heating device that may be applied as a component of the eye glasses or as a part of a headset. The heating device may be provided with a power to heat a moisturized pad that is immersed with liquid solution of the medicinal herbal composition such that the application of the eye lids and the surrounding areas of the eyes.

Specifically, in a preferred embodiment the invention discloses an apparatus for applying medication to eyes. The apparatus comprises a frame for securely holding a medication pad to eyes wherein the frame further includes a controllable heating element for heating the medication pad. The medication pad further comprises a moisturized compartment containing moisturized extracts of herbal medication for generating and applying a medication steam to the eyes when the heating element is controlled to apply heat to the medication pad. In another embodiment, in the medication pad the extracts of herbal medication further comprises extracts of goji berry and chrysanthemum. In another embodiment, the frame further comprises a battery controlled by a controller to provide power to the controllable heating element for heating the medication pad. In another embodiment, the frame further comprises a rechargeable battery controlled by a controller to provide power to the controllable heating element for heating the medication pad. In another embodiment, the frame further comprises a rechargeable battery that is rechargeable by solar power and controlled by a controller to provide power to the controllable heating element for heating the medication pad. In another embodiment, the frame further comprises a rechargeable battery that is rechargeable by solar power; and the frame further comprises a solar panel for absorbing solar energy to charge the rechargeable battery for heating the medication pad. In another embodiment, the frame further comprises a sensor to sensing a temperature applied to the medication pad and for sending a signal to the controller to turn off the heating element when a preset temperature is reached. In another embodiment, the frame further comprises a controller with a user interface display for allowing a user to enter control parameters to control the apparatus. In another embodiment, the frame further comprises a controller with a user interface display for allowing a user to enter control parameters to control the heating element. In another embodiment, the frame further comprises a controller with wireless signal transceiver to wireless control parameters to control the apparatus. In another embodiment, the frame further comprises a controller with wireless signal transceiver to communicate with a cellular phone to receive wireless control parameters to control the apparatus. In another embodiment, the frame further comprises a controller with wireless signal transceiver to communicate with a cellular phone to transmit and display operation conditions of the apparatus on the cellular phone.

These and other objects and advantages of the present invention will no doubt become obvious to those of ordinary skill in the art after having read the following detailed description of the preferred embodiment, which is illustrated in the various drawing figures.

### BRIEF DESCRIPTION OF THE DRAWINGS

FIG. 1 shows chemical compositions of Lycium barbarum polysaccharide (C₆H₁₂O₆) and betaine (C₅H₁₁NO₂).

FIG. 2 is a plan view of a medication pad comprising a medication compartment with a heating pad to heat up the moisturized medical compartment for eye application according to the invention.

FIG. 3 is a cross-section taken along line II-II of FIG. 1.

FIG. 4 shows a frame with a controllable heating element for heating the medication pad wherein the frame further includes a transceiver to display and control from a portable communication device such as a cellular phone.

### DETAILED DESCRIPTION OF THE INVENTION

The present invention is described in details based on the preferred embodiments illustrated in the accompanying drawings. Specifically, the present invention relates to an apparatus that comprises a frame mounted on the eyes to apply heat to a medication pad that contain medication to effectively improve the near triad causative of decrease in vision and dry eye.

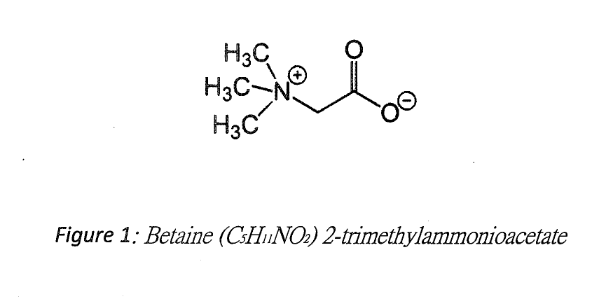

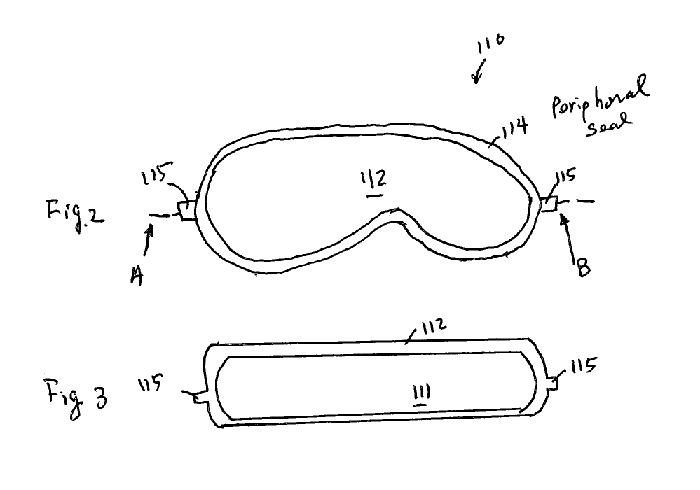

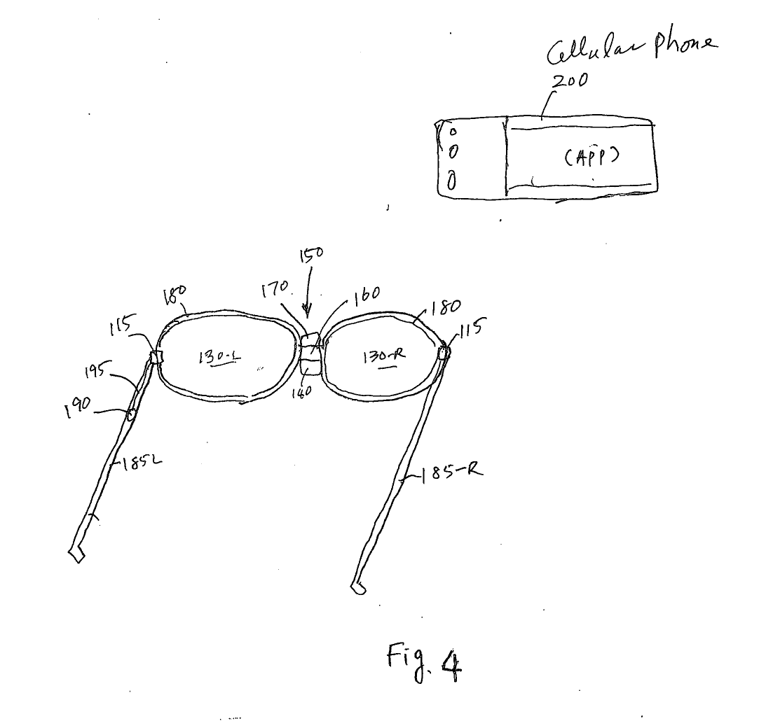

FIG. 1 illustrates a medication pad and FIG. 2 illustrates a cross sectional view along line A-B of the medication pad of this invention. The medication pad 110 is for mounting on a frame 100 shown in FIG. 3 for applying heat thereon for eye medication as a preferred embodiment of the present invention.

The medication pad 110 shown in FIG. 1 has a generally flat eye-mask shape of size enough to cover an area of both eyes that includes a moisturized medication compartment 111. The moisturized medication compartment is surrounded and held by a holder 112 holding therein the moisturized extracts of herbal medication in the compartment 111. The holder 112 is made of material that is suitable liar heat transfer and also moisture permeable to allow heated moisture to pass through the compartment holder 112 for applying the medicinal moisture to the eyes. As shown in FIG. 2, holder 112 is a fiat container formed of a closed space to hold the moisturized medication compartment steam generating member 111. The flat holder 112 has a first side that is to face the eyes of a wearer and a second side that is to face the opposite side with a heater to heat the medical pad wherein the heater is formed on the eyeglass frame as that shown in FIG. 3.

FIG. 3 shows a medication frame 100 with two mounting adapters 115 for mounting and supporting the medication pad 110 thereon. The medication frame 100 is formed with a shape similar to an eyeglass with a left and right supporting pads 130-L and 130-R. In one preferred embodiment, the supporting pads 130-L and 130-R have two sides. On the side facing the eyes is attached with two heating pads to heat up the medication pad 110 and on the opposite side of the heating pads are solar panels for exposing to the sun light to charge a rechargeable solar battery 140. The rechargeable solar battery is attached to a frame-bridge 150 and is charged by the solar energy received by the solar panels attached to the opposite side of the supporting pads 130-L and 130-R. Mounted on the frame-bridge 150 are also controller 160 for controlling the operation of the heating pads, and a transceiver 170 wherein the transceiver connected to antenna 180 formed as edge-frame surrounding the left and right supporting pads 130-L and 130-L. The battery, controller, and transceiver are controlled by a user controlling pad 190 mounted on a left or right frame legs 185-L and 185-R with wires 195 connected from the control pad 190 to the solar panels, the heating pads, the controller, and the transceiver to adjust the settings of these devices. The transceiver 170 further communicates with a wireless portable communication device such as a cellular phone 200 wherein the cellular phone further has functions to display and control the operation of the controller 160 and the medication pad 110 supported thereon so that the remote communication device 200 is enabled to adjust the operations of the heating pads, the solar panels and the communication with the user control pad 190 and the controller 160.

The moisturized medication compartment 111 contains moisturized medications that are extracts of medication herbal materials. The properties of the medication materials contained in the medication pad for the eyes of the present invention are as follows.

Both goji berry and chrysanthemum have been used frequently, over centuries, as a traditional Chinese medicine to brighten the eye. Laboratory research with rats has demonstrated that a mixture of liquid extracts of goji berry and chrysanthemum can protect the lens from oxidative damage.
[1] Goji Berry (Lycium barbarum L.) has been recognized as a general nutritive tonic, noted anti-aging herb, and eyesight-improving herbs for centuries. To keep healthy, promote good vision, and slow aging, people keen on nourishing life consume it on a daily basis by, for example, using it to make tea, medicinal rice wine, soup, congee, and sweets.
[2] [Botanical Description] Shrubs or large shrubs after cultivation, 1-3 m tall. Stems multiple, thick and strong, branchlets with longitudinal ribs, short spines without leaves and long spines with leaves and flowers, fruiting branch thin and long, generally apex nutant, bark greyish yellow, glabrous. Leaves alternate or fascicular in short branches; blade lanceolate or oblong lanceolate, 2-8 cm long, 0.5-3 cm wide, apex acuminate, base cuneate or narrowly cuneate extending to petiole, entire, upper surface dark green, below surface light green, glabrous. Flowers axillary, always 1 or 2-6 flowers fascicular on short branches, peduncle slender, calyx amphisperous, 4-5 mm long, apex 2-3 parted, lobes broadly ovate or ovate deltoid; corolla funnel shaped, tube 8 mm long, apex 5 lobed, lobes ovate, ca. 5 mm long, pink or light purple, with dark purple veins, a circle of pubescence above the stamen in the tube; stamens 5; pistil 1, ovary oblong, chambers 2, style linear, stigma head shape. Berry orbicular ovate, elliptic or broadly ovate, 8-20 mm long, 5-10 mm in diameter, red or orange red, peel fleshy. Seeds numerous, nearly round kidney shaped and flat, brownish yellow. Flowering: May to October, fruiting: July to November¹.
[3] As shown in the Chinese Materia Medica, it is sweet in taste and neutral in properties. It covers two channels of liver and kidney. Common uses and indications are blurred vision,
[4] Soreness and weakness of waist and knees, consumptive cough, light-headedness, and so on.
[5] [Chemical Composition] Lycium barbarum polysaccharide (C₆H₁₂O₆) and betaine (C₅H₁₁NO₂) as that shown in FIG. 1.
[6] [Pharmacology] Goji Berries have been reported to have various pharmaceutical properties, including osmoprotectant in the eye, reducing the risks of hypoglycemia, hypolipidemia and metabolic syndromes, immune enhancement, antioxidative, antitumor, anti-inflammatory, hepatoprotective and renal protective activities.
[7] [Nutritional Facts]² ² loan Gogoasa, Liana Alda, Maria Rada, Petru Negrea, Adina Negrea, Despina-Maria Bordean, Ariana Velciov, George Andrei Draghici, losif Gergen, Goji berries (Lycium barbarum) as a source of trace elements in human nutrition, Journal of Agroalimentary Processes and Technologies 2014, 20(4), 369-37
   a. Goji berries contain 19 amino acids the building blocks of protein including eight that are essential for life.
   b. Goji berries contain 21 trace minerals, including germanium, an anti-cancer trace mineral rarely found in foods.
   c. Goji berries contain more protein than whole wheat (13 percent).
   d. Goji berries contain a complete spectrum of antioxidant carotenoids, including beta-carotene (a better source than even carrots!) and zeaxanthin (protects the eyes). Goji berries are the richest source of carotenoids of all known foods.
   e. Goji berries contain Vitamin C at higher levels than even those found in oranges.
   f. Goji berries contain B-complex vitamins, necessary for converting food into energy.
   g. Goji berries contain Vitamin E (very rarely found in fruits, only in grains and seeds).
   h. Goji berries contain Beta-Sitosterol, an anti-inflammatory agent. Beta-Sitosterol also lowers cholesterol and has been used to treat sexual impotence and prostate enlargement.
   i. Goji berries contain essential fatty acids, which are required for the body's production of hormones and for the smooth functioning of the brain and nervous system.
   j. Goji berries contain Cyperone, a sesquiterpene that benefits the heart and blood pressure, alleviates menstrual discomfort, and has been used in the treatment of cervical cancer.
   k. Goji berries contain Solavetivone, a powerful anti-fungal and anti-bacterial compound.
   l. Goji berries contain Physalin, a natural compound that is active against all major types of leukemia. It has been shown to increase splenic natural killer cell activity in normal and tumor-bearing mice, with broad-spectrum anti-cancer effect. It has also been used as a treatment for hepatitis B.
   m. Goji berries contains Betaine, which is used by the liver to produce choline, a compound that calms nervousness, enhances memory, promotes muscle growth, and protects against fatty liver disease. Betaine also provided methyl groups in the body's energy reactions and can help reduce levels of homocysteine, a prime risk factor in heart disease. It also protects DNA.
[8] [Clinical Trials]
   a. Goji Berry has betaine and lycium barbarum polysaccharide, can be used as osmoprotectant in the eye, according to clinical trials of cells and animals to support 10 mM betaine compared with placebo on the 14, 21, 35 days were significantly reduced the dry eye's symptom.³ ³ Chen W I, Zhang X, Li J, Wang Y, Chen Q, Hou C, Garrett Q., Efficacy of osmoprotectants on prevention and treatment of murine dry eye, Invest Ophthalmol Vis Sci. 2013 Sep. 19; 54(9):6287-97. doi: 10.1167/iovs.13-12081.
   b. Betaine functions as an osmoprotectant to suppress inflammatory response via TRPV1 pathway in HCECs exposed to hyperosmotic stress. Osmoprotectants may have efficacy in reducing innate inflammation in dry eye disease⁴. ⁴ Xia Hua, Zhitao Su, Ruzhi Deng, Jing Lin, De-Quan Li, and Stephen C. Pflugfelder, Effects of L-carnitine, Erythritol and Betaine on Pro-inflammatory Markers in Primary Human Corneal Epithelial Cells Exposed to Hyperosmotic Stress, Curr Eye Res. 2015 July; 40(7): 657-667.
   c. Lycium barbarum polysaccharide and betaine present in goji berry may have important effects in alleviating dry eye disease induced by oxidative stress and inflammation⁵ ⁵ Chien K J, Horng C T, Huang Y S, Hsieh Y H, Wang C J, Yang J S, Lu C C, Chen F A, Effects of Lycium barbarum (goji berry) on dry eye disease in rats, Molecular Medicine Reports 17: 809-818, 2018
[9] [Safety] The goji berry has a long tradition of use as a medicinal and food plant in East Asia, in particular in China. It is a safe, functional food with beneficial effects on eyes.
[10] Chrysanthemum (Chrysanthemum morifolium Ramat) is an edible medicinal herb. As an important medicinal and edible plant, Chrysanthemum morifolium has a complex chemical composition with extensive uses, and it has been widely used in food, tea, and pharmaceutical products. The active ingredients such as flavonoids, terpenes, and organic acids isolated from the plant possess a variety of pharmacological effects, for example, antitumor, reducing blood fat, antimicrobial, analgesic, immunomodulatory, and antifatigue activities. The paper focused on reviewing the domestic and international studies on chemical constituents and pharmacological effects of C. morifolium during the past two decades, so as to provide a reference for further research and development of the plant resources⁶. ⁶ QU Lu, WANG Tao, DONG Yong-zhe, ZHANG Jing-hui, ZHANG Yi,Research progress on chemical constituents of Chrysanthemum morifoliuin and their pharmacologic activities, Drug Evaluation Research February 2015 (38,1)
[11] [Botanical Description] It is a perennial herb, 60 to 150 cm high. Erect stems are pubescent and branched or unbranched. Alternate leaves are with a short handle; leaf blade is from ovate to lanceolate, 5 to 15 cm long, pinnatilobate or pinnatifid, and with cuneate base. Capitulum is 2.5 to 20 cm in diameter and in various sizes. Single or a few capitulums gather on the top of the stems. Bracts have multiple layers. The bar-type outer layer is green, pubescent outside, and with membranous margin. And ligulate flowers come in white, red, purple or yellow. Achenes are not developed. Flowering time is from September to November. Perennial herbs, 60-150 cm tall. Stem erect, branched or unbranched, pubescent. Leaves alternate; with short petioles; leaves ovate or lanceolate, 5-15 cm long, pinnate, argin thick serrated or parted, base cuneate, lower part white pubescent, apex of lobes round or blunt; with petioles. Capitulums 2.5-20 cm in diameter, ranging in size, solitary or several clustered on the top of stem; phyllary with multiple layers, outer layer of phyllary green, linear, margin membranaceous, outside pubescent. Tubular flowers yellow; ligulate flowers white, red, purple or yellow. Achenes sterile.
[12] As shown in the Chinese Materia Medica, it is sweet and bitter in taste and slightly cold in medicinal properties. It covers two meridians of lung and liver. Common uses and indications include wind-heat common cold, fever and headache, vertigo, red sore eyes, blurred eyesight, toxicity of detumescence, deep-rooted boil and pyogenic infections.
[13] [Chemical Composition] Chlorogenic acid (C₁₆H₁₈O₉₎, Luteolin (C₂₁H₂₀O₁₁), and 3,5-O-Dicaffeoylquinic acid (C₂₅H₂₄O₁₂₎.
[14] [Pharmacology] Chrysanthemum has been reported to have various pharmaceutical properties of immunomodulatory, vasodilatation, antitumor, antibacterial, anti-inflammatory, anti-oxidative, anti-microbial, analgesic, lowing blood fat and excreting lead.
[15] [ Safety] The chrysanthemum has a long tradition of use as a medicinal, food and tea plant in East Asia, in particular in China. It is a safe, functional food with beneficial effects on eyes.

A dry eye herbal composition, comprising about 40-60% by weight of Goji berry, about 60%-40% by weight of Chrysanthemum, alleviates dry eye symptoms.

Clinical Study

Both of efficacy and safety were evaluated in this clinical study.

A total of 40 DED subjects (80 eyes) were randomized into a treatment group of 20 subjects (40 eyes, 9 males and 11 females with average age of 44.35±3.73) and a control group of 20 cases (40 eyes 8 males and 12 females with average age of 44.7±3.3). Medicinal herbal composition with the steam-generating heating device was used in the treatment group, whereas dextran-70 eye drops were used in the control group. There were no statistical differences between the two groups in terms of gender, age and duration (P>0.05).

Diagnostic Criteria

This is based on subjective symptoms and eye examinations. Subjective symptoms include dry eyes, eyestrain, foreign body sensation, discharges, itching, light sensitivity and unexplained discomfort of the eyes. Eye examinations include Tear volume of Schirmer I Test (SIT) <10 mm in 5 min and Break-up Time (BUT) of tear film ≤10 s.

Methods

Control Group: dextran-70 eye drops, 1˜2 drops each time, 3 times a day. Treatment Group: Herbal atomizer fumigation with 200 ml steam of Goji Berry 10 g, Chrysanthemum 5 g, twice a day, 30 min each time, maintain 93.2° F.˜109.4° F. temperature. 30 days after treatment for the statistical analysis.

Therapeutic Efficacy Criteria
1. The degree of the clinical symptoms improvement
2. Schirmer's I test, performed without anaesthesia (≤5 mm in 5 minutes)
Significant Efficacy: Significant alleviation of major symptoms, SIT>10 mm/5 min, and BUT>10 s.
Efficacy: Somewhat alleviation of major symptoms, SIT is 5-10 mm/5 min and BUT is 5-10 s.
No Efficacy: No improvement in symptoms, SIT<5 mm/5 min and BUT<5 s.

In the treatment group of 20 patients (40 eyes), 10 eyes were cured, 14 eyes showed significant efficacy, 12 eyes efficacy, 4 eyes have no efficacy. Thus, the total effective rate was 90%; in the control group of 20 patients (40 eyes), 6 eyes were cured, 10 eyes showed significant efficacy, 14 eyes showed efficacy, 10 eyes with no efficacy. Thus, for the control group, the total effective rate was 75%. P<0.05 indicates a statistical significance.

Results

The total effective rate in the treatment group was 90%, versus 75% in the control group, showing a statistically significant difference. There were no side effects in either group and no patient withdrew from the study.

### EXAMPLES

### Example 1

In this example, a medicament for treating dry eye is prepared according to the following weight proportions:

60% Goji berry, 40% of chrysanthemum.

Specific usage, the medicine was boiled with 200 ml water, a towel was soaked in the liquid, allowed to cool to 40° F. and then placed over the eyes. The towel was left on the eyes for 30 minutes, keeping the skin temperature around the eyes and eyes at 93.2° F.˜109.4° F. This procedure was repeated, according to the severity of symptoms, 1-3 times a day.

### Example 2

In this example, a medication for treating dry eye is prepared according to the following weight proportions:

Medlar 50, 50% of chrysanthemum.

[>Usable beauty steam machine, placed in the container of the pharmaceutical composition, the top of the steam through the combination of drugs fumigation eyes, for 30 minutes and 93.2° F.˜109.4° F. temperature, according to the severity of symptoms, 1-3 times a day. << does not make sense.

### Example 3

In this example, a medication for treating dry eye is prepared according to the following weight proportions:

Goji berry 1.5 g and Chrysanthemum 1.5 g

A heating steam is adapted to the powder of the medicinal herbal composition. The upper layer is protected with a non-woven fabric, the above herbal composition is placed, and an eye mask is formed from the self-heating steam generating layer, the non-woven fabric underlying layer and the oxygen barrier pouch. When used, open the oxygen barrier plastic bag, wear eye mask, so that the hot vapor through the combination of drugs, maintaining 30 minutes and 93.2° F.˜109.4° F. temperature. According to the severity of symptoms, application of 1-3 times in a day may be required.

Conclusion

This invention was effective and safe in alleviating DED symptoms.

FIG. 3 shows a frame 100 for securely holding the medication pad 110 with a moisturized medication compartment 111 held by the holder 112. The frame 100 holds the medication pad 111 to apply the medication to the eyes wherein the frame 100 further includes a controllable heating element for heating the medication pad. The medication pad further comprises a moisturized compartment containing moisturized extracts of herbal medication for generating and applying a medication steam to the eyes when the heating element is controlled to apply heat to the medication pad. In another embodiment, in the medication pad the extracts of herbal medication further comprises extracts of goji berry and chrysanthemum. In another embodiment, the frame further comprises a battery controlled by a controller to provide power to the controllable heating element for heating the medication pad. In another embodiment, the frame further comprises a rechargeable battery controlled by a controller to provide power to the controllable heating element for heating the medication pad. In another embodiment, the frame further comprises a rechargeable battery that is rechargeable by solar power and controlled by a controller to provide power to the controllable heating element for heating the medication pad. In another embodiment, the frame further comprises a rechargeable battery that is rechargeable by solar power; and the frame further comprises a solar panel for absorbing solar energy to charge the rechargeable battery for heating the medication pad. In another embodiment, the frame further comprises a sensor to sensing a temperature applied to the medication pad and for sending a signal to the controller to turn off the heating element when a preset temperature is reached. In another embodiment, the frame further comprises a controller with a user interface display for allowing a user to enter control parameters to control the apparatus. In another embodiment, the frame further comprises a controller with a user interface display for allowing a user to enter control parameters to control the heating element. In another embodiment, the frame further comprises a controller with wireless signal transceiver to wireless control parameters to control the apparatus. In another embodiment, the frame further comprises a controller with wireless signal transceiver to communicate with a cellular phone to receive wireless control parameters to control the apparatus. In another embodiment, the frame further comprises a controller with wireless signal transceiver to communicate with a cellular phone to transmit and display operation conditions of the apparatus on the cellular phone.

Though the invention has been described with respect to specific preferred and alternative embodiments, many additional variations and modifications will become apparent to those skilled in the art upon reading the present application. Thus it is the intention that the appended claims be interpreted as broadly as possible in view of the prior art to include all such variations and modifications.

I claim:

1. An apparatus for applying medication to eyes comprising:
a frame for securely holding a medication pad to eyes wherein the frame further includes a controllable heating element for heating the medication pad; and
the medication pad further comprises a moisturized compartment containing moisturized extracts of herbal medication for generating and applying a medication steam to the eyes when the heating element is controlled to apply heat to the medication pad.

2. The apparatus for applying medication to the eyes of claim 1 wherein:
the extracts of herbal medication further comprises extracts of goji berry and chrysanthemum.

3. The apparatus for applying medication to the eyes of claim 1 wherein:
the frame further comprises a battery controlled by a controller to provide power to the controllable heating element for heating the medication pad.

4. The apparatus for applying medication to the eyes of claim 1 wherein:
the frame further comprises a rechargeable battery controlled by a controller to provide power to the controllable heating element for heating the medication pad.

5. The apparatus for applying medication to the eyes of claim 1 wherein:
the frame further comprises a rechargeable battery that is rechargeable by solar power and controlled by a controller to provide power to the controllable heating element for heating the medication pad.

6. The apparatus for applying medication to the eyes of claim 1 wherein:
the frame further comprises a rechargeable battery that is rechargeable by solar power; and
the frame further comprises a solar panel for absorbing solar energy to charge the rechargeable battery for heating the medication pad.

7. The apparatus for applying medication to the eyes of claim 1 wherein:
the frame further comprises a sensor to sensing a temperature applied to the medication pad and for sending a signal to the controller to turn off the heating element when a preset temperature is reached.

8. The apparatus for applying medication to the eyes of claim 1 wherein:
the frame further comprises a controller with a user interface display for allowing a user to enter control parameters to control the apparatus.

9. The apparatus for applying medication to the eyes of claim 1 wherein:
the frame further comprises a controller with a user interface display for allowing a user to enter control parameters to control the heating element.

10. The apparatus for applying medication to the eyes of claim 1 wherein:
the frame further comprises a controller with wireless signal transceiver to wireless control parameters to control the apparatus.

11. The apparatus for applying medication to the eyes of claim 1 wherein:
the frame further comprises a controller with wireless signal transceiver to communicate with a cellular phone to receive wireless control parameters to control the apparatus.

12. The apparatus for applying medication to the eyes of claim 1 wherein:
the frame further comprises a controller with wireless signal transceiver to communicate with a cellular phone to transmit and display operation conditions of the apparatus on the cellular phone.
